(19) United States
(12) Patent Application Publication
(10) Pub. No.: US 2025/0303155 A1
(43) Pub. Date: Oct. 2, 2025

(54) Applying Alternating Electric Fields to Multiple Regions of Interest Within a Body

(71) Applicant: Novocure GmbH, Baar (CH)

(72) Inventors: Ariel NAVEH, Haifa (IL); Ari M. FRANK, Haifa (IL)

(73) Assignee: Novocure GmbH, Baar (CH)

(21) Appl. No.: 19/092,778

(22) Filed: Mar. 27, 2025

Related U.S. Application Data
(60) Provisional application No. 63/571,535, filed on Mar. 29, 2024.

(51) Int. Cl. A61N 1/36; A61N 1/04

(52) U.S. Cl. CPC A61N 1/36002; A61N 1/0408; A61N 1/0476; A61N 1/36034

(57) ABSTRACT

With certain types of cancer and/or in certain subjects, situations may arise in which the cancer is not confined to a single, well localized position. For example, an individual subject may have two distinct tumor regions that are located a few cm apart from each other. When alternating electric fields (e.g., TTFields) are used to treat such tumors, it can be difficult to ensure that both tumors receive a sufficiently high electric field strength using the prior art approach that relies on only four transducer arrays to induce the TTFields. The embodiments described herein employ one or more additional electrode assemblies to shape the path of the electric field within the subject's body, so that the strength of the electric field will be above the therapeutic threshold in all the regions of interest (e.g., in both of the two distinct tumor regions).

### CROSS REFERENCE TO RELATED APPLICATIONS

This application claims the benefit of U.S. Provisional Application 63/571,535, filed Mar. 29, 2024, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 50-500 kHz, 75-300 kHz, or 150-250 kHz). FIG. 1 depicts the prior art Optune® system, which delivers 200 kHz TTFields to patients via four electrode assemblies (also referred to as “transducer arrays”) that are placed on the patient's skin near the tumor. The transducer arrays are arranged in two pairs (or “channels”), with one pair of transducer arrays 10L, 10R positioned to the left and right of the tumor, and the other pair of transducer arrays 10A, 10P positioned anterior and posterior to the tumor. Each transducer array is connected via a multi-wire cable to an AC signal generator 20.

Optune's AC signal generator (a) sends an AC current through the anterior/posterior (A/P) pair of transducer arrays for 1 second, which induces an electric field with a first direction through the tumor; then (b) sends an AC current through the left/right (L/R) pair of arrays for 1 second, which induces an electric field with a second direction through the tumor; then repeats steps (a) and (b) for the duration of the treatment.

### SUMMARY OF THE INVENTION

One aspect of the invention is directed to a first method of applying alternating electric fields to both a first region of interest within a subject's body and a second region of interest within the subject's body. The first method comprises (a) applying an alternating voltage between (i) a first electrode assembly positioned at a first location on or in the subject's body and (ii) a plurality of second electrode assemblies, each of which is positioned at a respective second location on or in the subject's body. The first electrode assembly and a first one of the second electrode assemblies are positioned on opposite sides of the first region of interest. The first electrode assembly and a second one of the second electrode assemblies are positioned on opposite sides of the second region of interest. The first one of the second electrode assemblies and the second one of the second electrode assemblies are spaced apart from each other by at least 5 cm, and the alternating voltage has a frequency between 50 kHz and 1 MHz.

Some instances of the first method further comprise (b) applying an alternating voltage between a third electrode assembly positioned at a third location on or in the subject's body and a fourth electrode assembly positioned at a fourth location on or in the subject's body. In these instances, the third electrode assembly is positioned between the first electrode assembly and the first one of the second electrode assemblies, and the fourth electrode assembly is positioned between the first electrode assembly and the second one of the second electrode assemblies. Optionally, in these instances, step (a) and step (b) are repeated in an alternating sequence at least 100 times.

Some instances of the first method further comprise (b) applying an alternating voltage between a third electrode assembly positioned at a third location on or in the subject's body and a fourth electrode assembly positioned at a fourth location on or in the subject's body. In these instances, the third electrode assembly is positioned radially between the first electrode assembly and the first one of the second electrode assemblies, and the fourth electrode assembly is positioned radially between the first electrode assembly and the second one of the second electrode assemblies. Optionally, in these instances, step (a) and step (b) are repeated in an alternating sequence at least 100 times.

Some instances of the first method further comprise (b) applying an alternating voltage between (i) a third electrode assembly positioned at a third location on or in the subject's body and (ii) a plurality of fourth electrode assemblies, each of which is positioned at a respective fourth location on or in the subject's body. In these instances, the third electrode assembly is positioned between the first one of the second electrode assemblies and the second one of the second electrode assemblies, and the first electrode assembly is positioned between a first one of the fourth electrode assemblies and a second one of the fourth electrode assemblies. Optionally, in these instances, step (a) and step (b) are repeated in an alternating sequence at least 100 times.

Some instances of the first method further comprise (b) applying an alternating voltage between (i) a third electrode assembly positioned at a third location on or in the subject's body and (ii) a plurality of fourth electrode assemblies, each of which is positioned at a respective fourth location on or in the subject's body. In these instances, the third electrode assembly is positioned radially between the first one of the second electrode assemblies and the second one of the second electrode assemblies, and the first electrode assembly is positioned radially between a first one of the fourth electrode assemblies and a second one of the fourth electrode assemblies. Optionally, in these instances, step (a) and step (b) are repeated in an alternating sequence at least 100 times.

In some instances of the first method, the subject's body includes a third region of interest, the first electrode assembly and a third one of the second electrode assemblies are positioned on opposite sides of the third region of interest, and all of the second electrode assemblies are spaced apart from each other by at least 5 cm or at least 8 cm.

In some instances of the first method, the first one of the second electrode assemblies and the second one of the second electrode assemblies are spaced far enough apart from each other to create a low-intensity-field zone between the first region of interest and the second region of interest. In some instances of the first method, the first one of the second electrode assemblies and the second one of the second electrode assemblies are spaced apart from each other by at least 8 cm.

Some instances of the first method further comprise, prior to step (a): positioning the first electrode assembly at the first location on or in the subject's body; and positioning each of the plurality of second electrode assemblies at the respective second location on or in the subject's body.

In some instances of the first method, the alternating voltage has a frequency between 75 kHz and 300 kHz.

Another aspect of the invention is directed to a first apparatus for applying alternating electric fields to both a first region of interest within a subject's body and a second region of interest within the subject's body. The first apparatus comprises an AC signal generator, a first electrode assembly, and a plurality of second electrode assemblies. The AC signal generator operates at a frequency between 50 kHz and 1 MHZ, and the AC signal generator applies a first output voltage across a first output pin and a second output pin. The first electrode assembly is configured to adhere to the subject's body and is connected to the first output pin via a first cable. And each of the plurality of second electrode assemblies is configured to adhere to the subject's body and is connected to the second output pin via a respective one of a plurality of second cables. All of the second electrode assemblies are wired in parallel, and the plurality of second cables are configured so that the plurality of second electrode assemblies can be spaced apart from each other by at least 5 cm.

In some embodiments of the first apparatus, the AC signal generator applies a second output voltage across a third output pin and a fourth output pin. In these embodiments, the apparatus further comprises a third electrode assembly and a fourth electrode assembly. The third electrode assembly is configured to adhere to the subject's body and is connected to the third output pin via a third cable, and the fourth electrode assembly is configured to adhere to the subject's body and is connected to the fourth output pin via a fourth cable. The AC signal generator is configured to (a) apply the first output voltage across the first output pin and the second output pin, (b) apply the second output voltage across the third output pin and the fourth output pin, and repeat (a) and (b) in an alternating sequence at least 100 times.

In some embodiments of the first apparatus, the AC signal generator applies a second output voltage across a third output pin and a fourth output pin. In these embodiments, the apparatus further comprises a third electrode assembly and a plurality of fourth electrode assemblies. The third electrode assembly is configured to adhere to the subject's body and is connected to the third output pin via a third cable. And each of the plurality of fourth electrode assemblies is configured to adhere to the subject's body and is connected to the fourth output pin via a respective one of a plurality of fourth cables. All of the fourth electrode assemblies are wired in parallel. The plurality of fourth cables are configured so that the plurality of fourth electrode assemblies can be spaced apart from each other by at least 5 cm. And the AC signal generator is configured to (a) apply the first output voltage across the first output pin and the second output pin, (b) apply the second output voltage across the third output pin and the fourth output pin, and repeat (a) and (b) in an alternating sequence at least 100 times.

In some embodiments of the first apparatus, the AC signal generator operates at a frequency between 75 kHz and 300 kHz.

### BRIEF DESCRIPTION OF THE DRAWINGS

FIG. 1 depicts a prior art apparatus for performing Tumor Treating Fields (TTFields) therapy.

FIG. 2 is a schematic diagram of an apparatus for performing TTFields therapy in a portion of the subject's body that includes two regions of interest (ROIs).

FIG. 3 is a schematic diagram of another apparatus for performing TTFields therapy in a portion of the subject's body that includes two ROIs.

FIG. 4 is a schematic diagram of another apparatus for performing TTFields therapy in a portion of the subject's body that includes two ROIs.

FIG. 5 is a schematic diagram of another apparatus for performing TTFields therapy in a portion of the subject's body that includes two ROIs.

FIGS. 6A and 6B depict simulation results for the FIG. 1 and FIG. 5 configurations, respectively.

FIG. 7 is a schematic diagram of another apparatus for performing TTFields therapy in a portion of the subject's body that includes three ROIs.

FIG. 8 is a schematic diagram of another apparatus for performing TTFields therapy in a portion of the subject's body that includes two ROIs, with an exclusion zone between the two ROIs.

FIG. 9 is a flowchart of an example procedure of applying alternating electric fields to multiple ROIs within a subject's body.

Various embodiments are described in detail below with reference to the accompanying drawings, wherein like reference numerals represent like elements.

### DESCRIPTION OF THE PREFERRED EMBODIMENTS

The prior art configuration of transducer arrays (in which one pair of transducer arrays is positioned to the left and right of the tumor, and the other pair of transducer arrays is positioned anterior and posterior to the tumor) is very well-suited for treating a tumor that has a single, well localized position. But with certain types of cancer and/or in certain subjects, situations may arise in which the cancer is not confined to a single, well localized position. For example, an individual subject may have two distinct tumor regions that are located a few cm apart from each other. In this situation, it can sometimes be difficult to ensure that both tumors receive a sufficiently high electric field strength (e.g., >1 V/cm, e.g., 1-10 V/cm) using the prior art approach that relies on L/R and A/P transducer arrays. For if the transducer arrays are positioned to optimize the field strength in one of the tumors, the field strength in the second tumor may be below the therapeutic threshold value (e.g., 1 V/cm). And if the transducer arrays are positioned to optimize the field strength in the second tumor, the field strength in the first tumor may be below the therapeutic threshold value.

The embodiments described herein employ one or more additional electrode assemblies to shape the path of the electric field within the subject's body, so that the strength of the electric field will be above the therapeutic threshold in two or more regions of interest (e.g., in both of the tumors in the example of the previous paragraph).

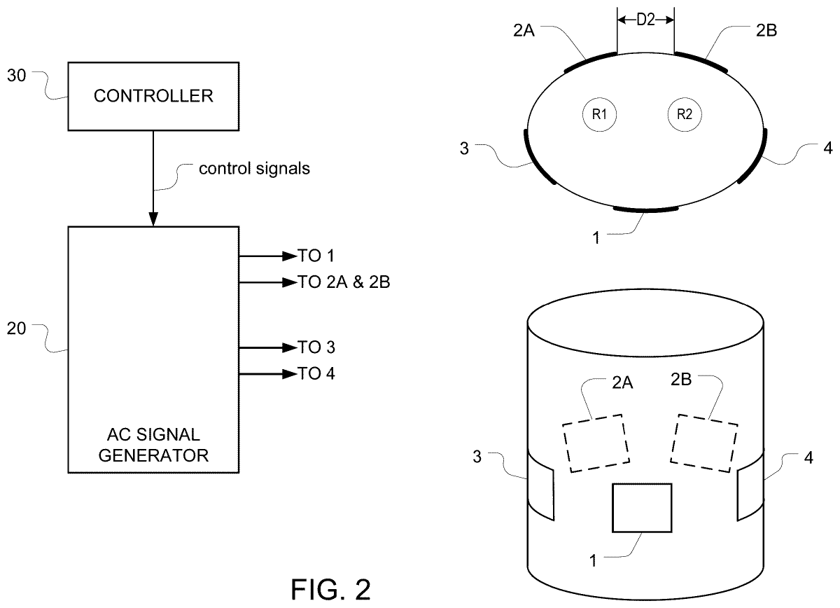

FIG. 2 is a schematic diagram of an apparatus for performing TTFields therapy in a portion of the subject's body that includes two non-contiguous regions of interest (R1 and R2). FIG. 2 includes a top view of a representation of subject's body part, and a front perspective view (with electrode assemblies that are hidden from view shown in dashed lines). In the FIG. 2 example, five electrode assemblies are positioned around the portion of the subject's body that includes the two regions of interest, R1 and R2. An anterior electrode assembly 1 is paired with two separate posterior electrode assemblies 2A and 2B that are positioned a distance D2 from each other. The first electrode assembly 1 and the electrode assembly 2A are located on opposite sides of the region of interest R1, and the electrode assembly 2B and the first electrode assembly 1 are on opposite sides of the region of interest R2. When an alternating voltage is applied between (i) the first electrode assembly 1 positioned at an anterior location on or in the subject's body and (ii) a plurality of second electrode assemblies 2A, 2B, each of which is positioned at a respective posterior location on or in the subject's body, electric fields are induce in both the regions of interest R1 and R2. Thus, through this positioning of electrode assemblies, the electrode assemblies 1, 2A, and 2B effectively define two separate paths that can be used to impose electric fields in both of the regions of interest R1 and R2 simultaneously.

The positions of the electrode assemblies 1, 2A, and 2B can be determined in advance to cover all of the regions of interest (e.g., all of the regions known to include a tumor). Determination of the positioning of electrode assemblies (in arrangements where multiple electrode assemblies form paths with a common/shared electrode assemblies) can be computed based on the locations of the regions of interest, and based on the type of electrode assemblies being used. In some embodiments, the electrode assemblies can be placed at specific pre-determined positions relative to each other. For example, the electrode assemblies 2A and 2B can be spaced apart from each other by at least 5 cm (represented as the D2 distance depicted in FIG. 2). This distance can result in electric field distributions that are sufficiently strong within the regions of interests R1 and R2.

As further shown in FIG. 2, the regions of interest R1 and R2 are also subjected to an electric field that has a side-to-side orientation (e.g., left-right orientation), achieved through the electrode assemblies 3 and 4. The electric fields can be applied in different directions in an alternating sequence by first (a) applying an alternating voltage between (i) the first electrode assembly 1 and (ii) both second electrode assemblies 2A, 2B for a first period of time (e.g., one second), which induces electric fields in both regions of interest R1 and R2 (b) applying an alternating voltage between the third electrode assembly 3 and the fourth electrode assembly 4 for a second period of time (e.g., one second), which induces electric fields in both regions of interest in a different direction, and repeating (a) and (b) in an alternating sequence e.g., at least 100 times. This will induce an alternating electric field in the regions of interest R1 and R2 whose direction changes repeatedly.

A wide variety of electrode assemblies (or regular electrodes) can be used in the implementations described herein, including but not limited to the electrode assemblies described in US 2023/0043071, entitled “Electrode Assembly for Applying Tumor Treating Fields (TTFields) that Include a Sheet of Graphite,” US 2021/0402179, entitled “Flexible Transducer Arrays with a Polymer Insulating Layer for Applying Tumor Treating Fields (TTFields),” and U.S. Pat. No. 8,715,203, entitled “Composite Electrode.” Each of these documents is incorporated herein by reference in its entirety. The electrode assemblies can be implemented to be of different sizes, based on specific needs of a particular situation.

The electrode assemblies depicted in FIG. 2, and in the other figures discussed below, can be secured to the skin using a conductive adhesive, such as conductive hydrogel adhesive or a conductive non-hydrogel polymer adhesive. For example, the electrode assemblies shown in the top view of FIG. 2 can be secured using an adhesive layer disposed to the contact surface of the electrode assemblies (i.e., the surfaces of the electrode assemblies facing the subject's skin). Alternatively or additionally, the electrode assemblies can be secured to the subject's body (to maintain their positions) using other electrode-securing mechanisms. For example, the electrode assemblies can be secured to the subject's body using bands wrapped over the electrode assemblies. Such bands can be manufactured from stretchable/flexible materials that can be used with body parts of different dimensions and contours, and may also be structured to operate to press the electrodes (or electrode assemblies) against the subject's body. Such stretchable/flexible materials may include breathable materials, e.g., cotton fabrics, through which sweat can permeate. The stretchable/flexible bands can be re-usable bands, in which case they can include fasteners (not shown) such as hook-and-loop fasteners (e.g., Velcro®) attached to opposite ends of the bands to allow adjustable fastening of the bands to form a closed loop to surround different dimensioned body parts. Other types of fastening mechanisms can be used, including various adhesive materials, belt-and-buckle fasteners, etc. Alternatively, bands to secure electrode assemblies to patients' body can be disposable bands such as bandages (e.g., band-aid type bandages) with a front surface (the surface contacting a subject's skin) at least partly covered with an adhesive layer so as to affix the bandage to the body part.

With continued reference to FIG. 2, the apparatus includes an AC signal generator 20 that is configured to generate and apply AC signals to the various electrode assemblies positioned on the subject's body. Note that while FIG. 2 depicts five electrode assemblies, the number of deployed electrode assemblies can vary. The AC signal generator 20 is configured to apply an AC voltage with a frequency of between 50 kHz and 1 MHz (e.g., 50-500 kHz, 75-300 kHz, or 150-250 kHz), with voltage levels that typically are on the order of 50-150 VRMS. One way to implement the AC signal generator 20 is described in U.S. Pat. No. 9,910,453, which is incorporated herein by reference in its entirety. Alternatively, a variety of alternative approaches for implementing the AC signal generator 20 that will be apparent to persons skilled in the relevant arts can be used.

The AC signal generator 20, and the apparatus in general, is controlled by a controller 30 (e.g., a processor-based controller). In some embodiments, the controller 30 controls the operation of the AC signal generator 20 based on operating temperature measurements (e.g., measured by thermistors positioned on the electrode assemblies) to adjust the AC signal that is applied to the electrode assemblies in order to maintain their temperatures below a safety threshold (e.g., 39° C.).

Accordingly, in some embodiments, an apparatus is provided for applying alternating electric fields to both a first region of interest within a subject's body and a second region of interest within the subject's body. The apparatus comprises an AC signal generator 20 that operates at a frequency between 50 kHz and 1 MHz (e.g., 50-500 kHz, 75-300 kHz, or 150-250 kHz) and a set of electrode assemblies 1-4. The AC signal generator 20 applies a first output voltage across a first output pin and a second output pin. A first electrode assembly 1 is connected to the first output pin via a first cable, and a plurality of second electrode assemblies 2A, 2B are connected to the second output pin via respective second cables (e.g., with all of the second electrode assemblies being wired in parallel). The plurality of second cables are configured so that the plurality of second electrode assemblies can be spaced apart from each other by at least 5 cm. When the AC signal generator 20 applies an AC voltage across the first output pin and the second output pin, the AC voltage is routed to the electrode assemblies 1-4, which are positioned on or in the subject's body. As a result, electric fields will be induced through the subject's body. Note that the various electrode assemblies within the plurality of second electrode assemblies do not necessarily have to be driven by the same AC signal. For example, the output of the AC signal generator 20 could be transformer coupled so that one of the second electrode assemblies 2A receives the full output voltage of the AC signal generator, while the other second electrode assembly 2B receives 75% of the full output voltage.

In some embodiments, the AC signal generator 20 applies a second output voltage across a third output pin and a fourth output pin. In these embodiments, a third electrode assembly 3 is connected to the third output pin, and a fourth electrode assembly 4 is connected to the fourth output pin via respective cables. In the example depicted in FIG. 2, the third electrode assembly 3 is positioned between the first electrode assembly 1 and the first one of the second electrode assemblies 2A, and the fourth electrode assembly 4 is positioned between the first electrode assembly 1 and the second one of the second electrode assemblies 2B. The AC signal generator 20 is configured to (a) apply the first output voltage across the first output pin and the second output pin, (b) apply the second output voltage across the third output pin and the fourth output pin, and repeat (a) and (b) in an alternating sequence e.g., at least 100 times.

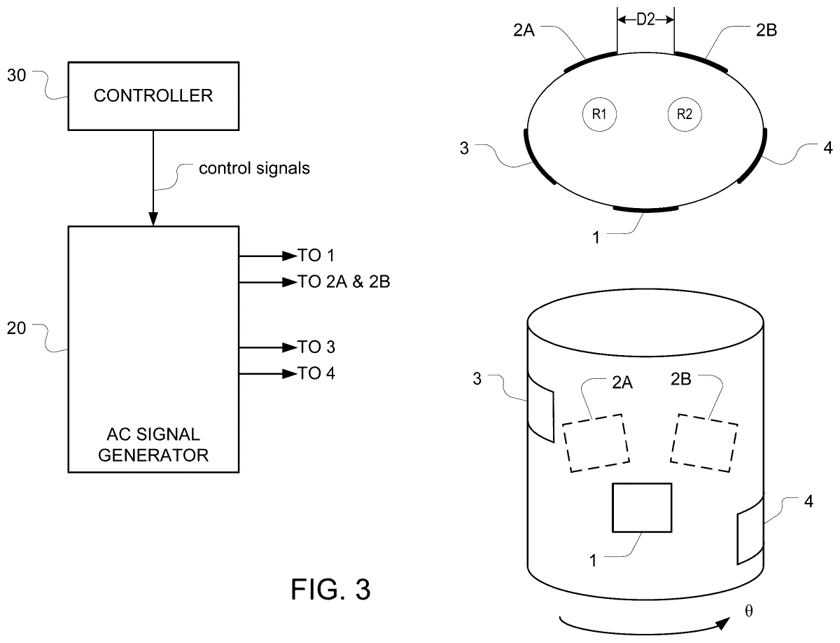

Note that the electrode assemblies need not all be positioned in a single planar region (e.g., a planar slice), but can be distributed in a three-dimensional volume, as depicted in FIG. 3. The arrangement of electrode assemblies in FIG. 3 is generally similar to that depicted FIG. 2, and as can be seen from the top view of the subject's body part, the electrode assemblies 1, 2A, 2B, 3, and 4 have similar radial positions as the positions of the electrode assemblies shown in FIG. 2. However, the electrode assemblies 3 and 4 in the FIG. 3 embodiment are positioned at different axial positions relative to the positions of the assemblies 2A, 2B, and 1. Thus, in a polar coordinate system in which each electrode assembly has a radial angle θ, as indicated in the front perspective view of FIG. 3, the electrode assembly 3 is positioned radially between the electrode assembly 1 and the electrode assembly 2A (i.e., the angle θ of electrode assembly 3 is between the angle of electrode assembly 1 and the angle of electrode assembly 2A). Additionally, in the example of FIG. 3, the electrode assembly 4 is positioned radially between the electrode assembly 1 and the electrode assembly 2B. The electrical connectivity of the electrode assemblies of FIG. 3 to the AC signal generator 20 is similar to that shown in FIG. 2, as is the timing for energizing those electrodes.

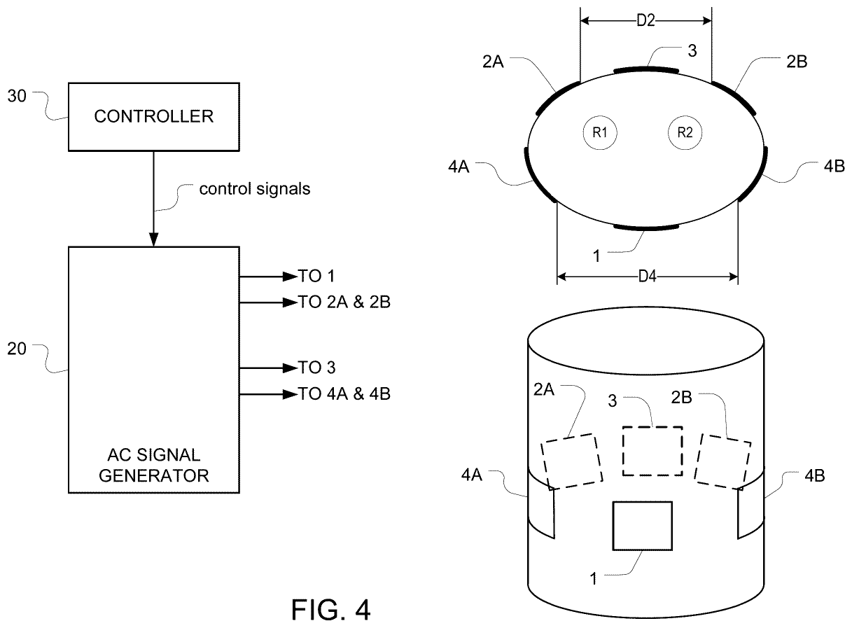

FIG. 4 is a diagram of another example apparatus for driving multiple electrode assemblies 1, 2A, 2B, 3, 4A, and 4B to induce alternating electric fields in different directions through more than one region of interest R1 and R2. When an alternating voltage is applied between (i) the first electrode assembly 1 positioned at an anterior location on or in the subject's body and (ii) a plurality of second electrode assemblies 2A, 2B, each of which is positioned at a respective posterior location on or in the subject's body, electric fields are induced in both the regions of interest R1 and R2 in respective directions simultaneously. This is similar to the situation described above in connection with FIG. 2. In addition, when an alternating voltage is applied between (i) the third electrode assembly 3 positioned at a posterior location on or in the subject's body and (ii) a plurality of fourth electrode assemblies 4A, 4B, each of which is positioned at a respective anterior location on or in the subject's body, electric fields are induced in both the regions of interest R1 and R2 in respective *different* directions simultaneously. This is similar to the situation described in this paragraph for the first and second electrode assemblies 1, 2A, 2B, but in a mirror image.

The controller 30 controls the AC signal generator 20 so that it (a) applies an alternating voltage between (i) a first electrode assembly 1 and (ii) a plurality of second electrode assemblies 2A, 2B, then (b) applies an alternating voltage between (i) a third electrode assembly 3 and (ii) a plurality of fourth electrode assemblies 4A, 4B, and then (c) repeats step (a) and step (b) in an alternating sequence (e.g., at least 100 times). The end result of applying alternating voltages in this sequence will be as follows: during step (a), electric fields will be induced in both the regions of interest R1 and R2 in respective directions simultaneously; during step (b), electric fields will be induced in both the regions of interest R1 and R2 in respective *different* directions simultaneously; and these two steps will repeat in an alternating sequence.

Note that the electrode assemblies in this FIG. 4 embodiment need not all be positioned in a single planar region (e.g., a planar slice), but can be distributed in a three-dimensional volume. This is similar to the situation described above in connection with FIG. 3. Thus, in the example depicted in FIG. 4, the third electrode assembly 3 is positioned radially between the second electrode assemblies 2A and 2B, and the first electrode assembly 1 is positioned radially between the fourth electrode assemblies 4A and 4B.

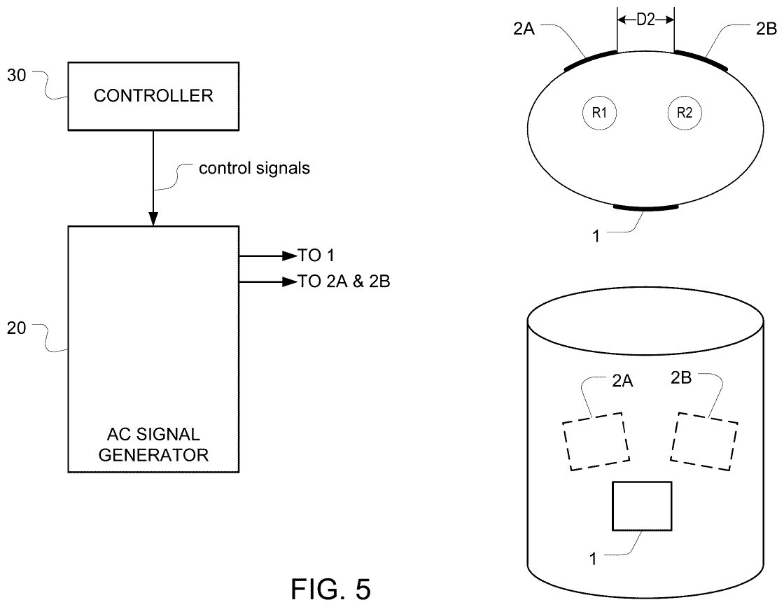

FIG. 5 shows an arrangement of electrode assemblies that are similar to those shown in FIG. 2, except that in FIG. 5 the electrode assemblies 3 and 4 are not included. The electric fields are only induced in both regions of interest R1 and R2 by applying an alternating voltage between (i) the first electrode assembly 1 and (ii) both second electrode assemblies 2A, 2B. As a result, the direction of the electric field within any given region of interest will not alternate back and forth between different directions, and will instead always be consistent.

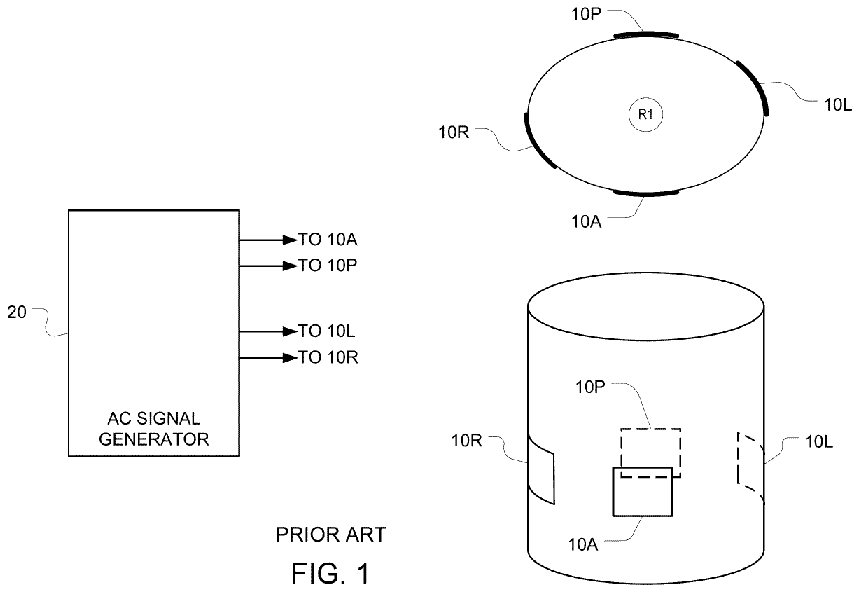

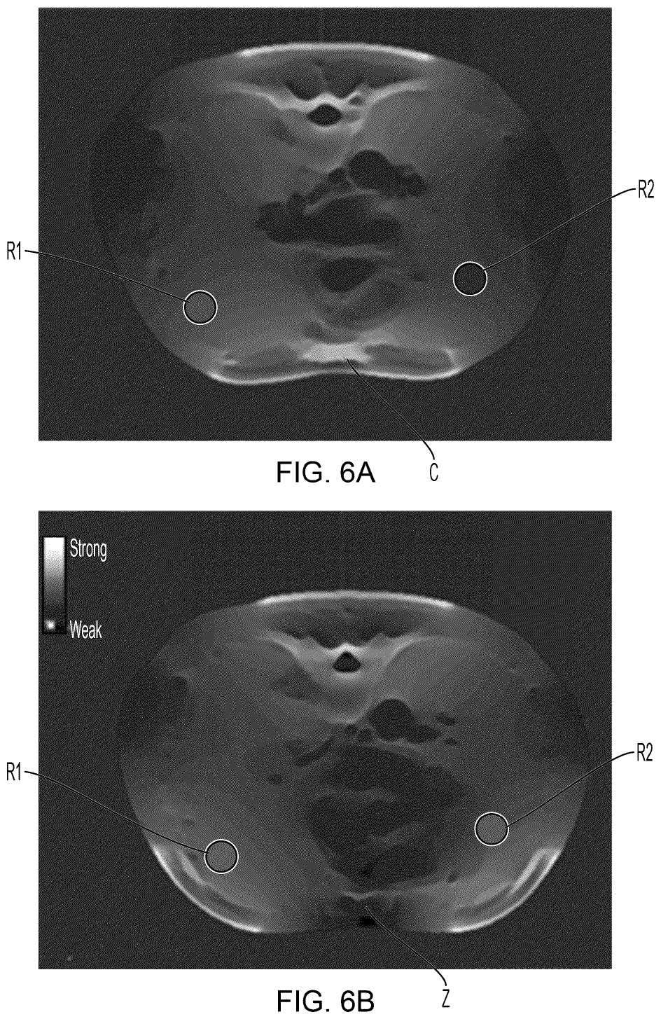

FIG. 6A is an intensity map that shows the electric field strength at various points in a horizontal slice of a model-subject's body for the prior art A/P electrode assembly configuration depicted in FIG. 1 (which has a single, centrally located anterior electrode assembly); and FIG. 6B is an intensity map that shows the electric field strength at various points in the same horizontal slice of the same model-subject's body for the electrode assembly configuration depicted in FIG. 5 (which has two anterior electrode assemblies spaced apart by about 10 cm). A review of FIG. 6A reveals that the electric field is quite strong just beneath the surface at the center of the model-subject's chest (i.e., at point C), and the electric field is significantly weaker at both of the regions of interest R1 and R2. It therefore follows that the FIG. 1 configuration for the A/P electrode assemblies is sub-optimal for treating tumors at those two regions of interest R1 and R2.

In contrast, a review of FIG. 6B reveals that the electric field is very weak just beneath the surface at the center of the model-subject's chest (i.e., at point Z), and the electric field is significantly stronger in both regions of interest R1 and R2. In addition, the electric field in FIG. 6B is stronger than the electric field in FIG. 6A in both regions of interest R1 and R2. It therefore follows that the FIG. 5 configuration (which uses a single posterior electrode assembly and two anterior electrode assemblies spaced apart by about 10 cm) is much better suited for treating tumors at those two regions of interest R1 and R2.

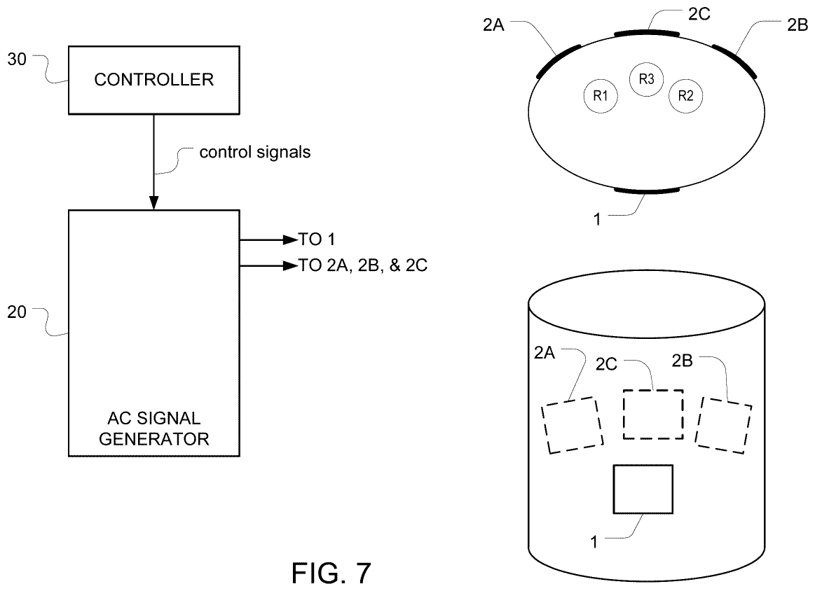

In the examples described above in connection with FIG. 2-5, there are only two regions of interest R1 and R2, and two posterior electrode assemblies 2A, 2B are used to establish paths for the electric field that travel through those regions of interest. But the concepts described above in connection with these figures can be extended to situations when there are more than two regions of interest (e.g., 3, 4, 5, or more ROIs). For example, FIG. 7 depicts an example where there are three regions of interest R1, R2, and R3. In this situation, three posterior electrode assemblies 2A, 2B, and 2C are used to establish three paths for the electric field that travel through all three of those regions of interest.

In the embodiments described above, the various electrode assemblies are arranged on or in a subject's body to induce electric fields in a plurality of regions of interest within the subject's body. This can be useful, for example, in situations where there are two or more distinct tumors in the subject's body, e.g., spaced apart by 5-10 cm.

In other situations, instead of striving to ensure that an electric field is induced in multiple regions of interest, it may be desirable to shape the electric fields to minimize the field strength within certain regions of the subject's body. For example, if a medical device is implanted in the subject's body, and if the alternating electric fields could disrupt the operation of the medical device, it would be desirable to shape the electric field to minimize the field strength in the immediate vicinity of the medical device.

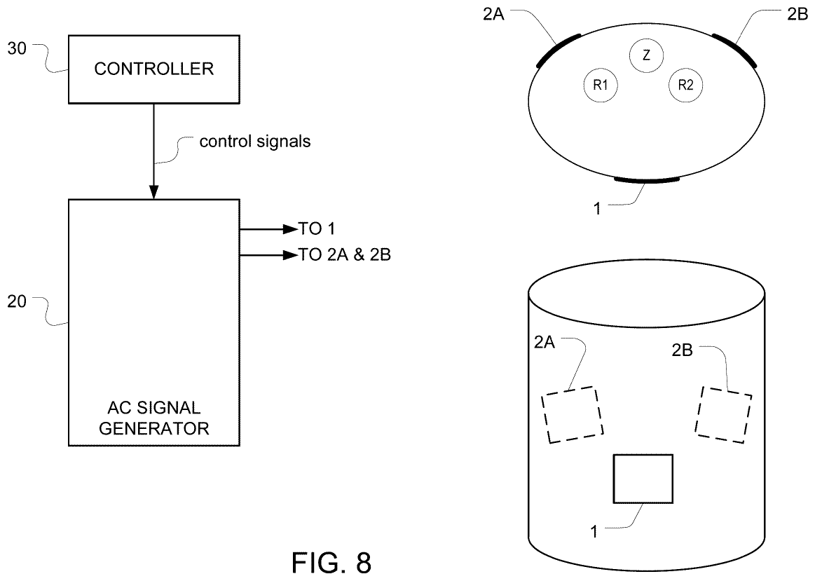

FIG. 8 depicts an example of this situation, in which Z represents an “exclusion zone” that should not be subjected to the electric fields. This situation can be addressed by positioning the electrode assemblies 1, 2A, 2B so that the electric field strength will be high enough to perform effective TTFields therapy on the regions of interest R1 and R2, but be low enough to prevent the implanted device (in zone Z) from malfunctioning. And the electric field strength simulation depicted in FIG. 6B confirms that spacing the electrode assemblies 2A, 2B apart from each other (e.g., by about 10 cm) will indeed create a low electric field strength “exclusion zone” in the region Z.

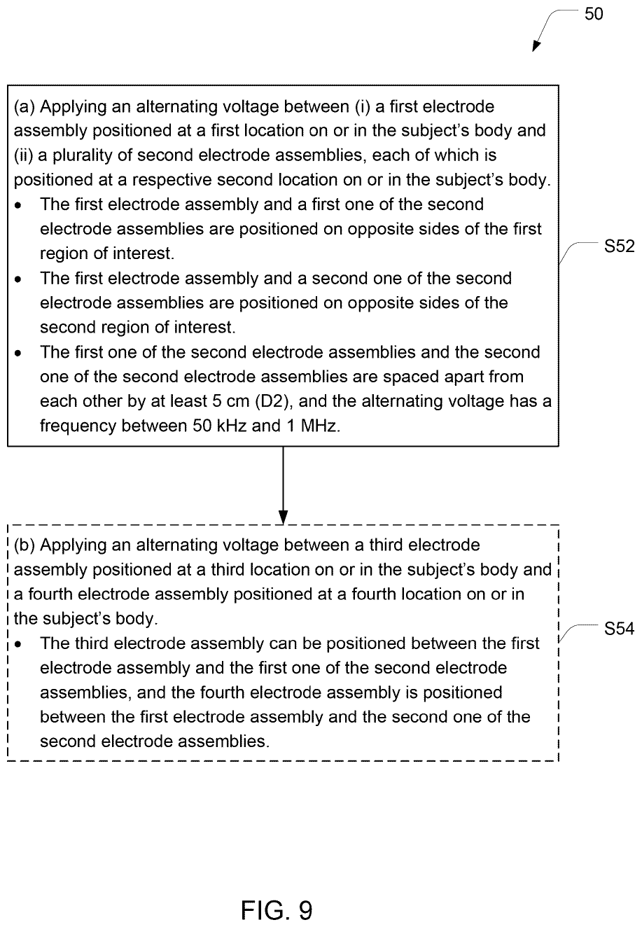

FIG. 9 is a flowchart of an example procedure 50 of applying alternating electric fields to both a first region of interest within a subject's body and a second region of interest within the subject's body. The method includes (a) applying (at S52) an alternating voltage between (i) a first electrode assembly positioned at a first location on or in the subject's body and (ii) a plurality of second electrode assemblies, each of which is positioned at a respective second location on or in the subject's body. The first electrode assembly and a first one of the second electrode assemblies are positioned on opposite sides of the first region of interest. The first electrode assembly and a second one of the second electrode assemblies are positioned on opposite sides of the second region of interest. The first one of the second electrode assemblies and the second one of the second electrode assemblies are spaced apart from each other by at least 5 cm, and the alternating voltage has a frequency between 50 kHz and 1 MHz (e.g., 50-500 kHz, 75-300 kHz, or 150-250 kHz).

In some embodiments, the procedure 50 can further include (b) applying (at S54) an alternating voltage between a third electrode assembly positioned at a third location on or in the subject's body and a fourth electrode assembly positioned at a fourth location on or in the subject's body. In such embodiments, the third electrode assembly can be positioned between the first electrode assembly and the first one of the second electrode assemblies, and the fourth electrode assembly is positioned between the first electrode assembly and the second one of the second electrode assemblies. In such embodiments, the procedure can further include repeating step (a) and step (b) in an alternating sequence at least 100 times.

In some embodiments, the procedure can further include (b) applying an alternating voltage between a third electrode assembly positioned at a third location on or in the subject's body and a fourth electrode assembly positioned at a fourth location on or in the subject's body. The third electrode assembly can be positioned radially between the first electrode assembly and the first one of the second electrode assemblies, and the fourth electrode assembly is positioned radially between the first electrode assembly and the second one of the second electrode assemblies. In such embodiments, the procedure can further include repeating step (a) and step (b) in an alternating sequence at least 100 times.

In some embodiments, the procedure can further include (b) applying an alternating voltage between (i) a third electrode assembly positioned at a third location on or in the subject's body and (ii) a plurality of fourth electrode assemblies, each of which is positioned at a respective fourth location on or in the subject's body. The third electrode assembly can be positioned between the first one of the second electrode assemblies and the second one of the second electrode assemblies, and the first electrode assembly can be positioned between a first one of the fourth electrode assemblies and a second one of the fourth electrode assemblies. In such embodiments, the procedure can further comprise repeating step (a) and step (b) in an alternating sequence at least 100 times.

In some embodiments, the procedure can further include (b) applying an alternating voltage between (i) a third electrode assembly positioned at a third location on or in the subject's body and (ii) a plurality of fourth electrode assemblies, each of which is positioned at a respective fourth location on or in the subject's body. The third electrode assembly can be positioned radially between the first one of the second electrode assemblies and the second one of the second electrode assemblies. The first electrode assembly can be positioned radially between a first one of the fourth electrode assemblies and a second one of the fourth electrode assemblies. In such embodiments, the procedure can further include repeating step (a) and step (b) in an alternating sequence at least 100 times.

In some embodiments, the subject's body can include a third region of interest. The first electrode assembly and a third one of the second electrode assemblies can be positioned on opposite sides of the third region of interest. All of the second electrode assemblies (2A-C) can be spaced apart from each other by at least 5 cm. In some examples, all of the second electrode assemblies can be spaced apart from each other by at least 8 cm.

In some embodiments, the first one of the second electrode assemblies and the second one of the second electrode assemblies can be spaced far enough apart from each other to create a low-intensity-field zone between the first region of interest and the second region of interest.

In some embodiments, the first one of the second electrode assemblies and the second one of the second electrode assemblies can be spaced apart from each other by at least 8 cm.

In some embodiments, the procedure 50 can further include, prior to step (a), positioning the first electrode assembly at the first location on or in the subject's body, and positioning each of the plurality of second electrode assemblies at the respective second location on or in the subject's body.

In some embodiments, the alternating voltage can have a frequency between 75 kHz and 300 kHz.

Finally, it is important to note that the usage of the identifiers (a), (b), (c), (d), etc. in the claims below does not imply a particular sequence in time for the corresponding steps. For while it is certainly possible that step (a) will precede step (b) in time, different sequencings of those steps are also possible, except in cases where a particular sequencing is inconsistent with the internal language of the various steps or with other language in the claims. For example, a step labeled (b) could precede a step labeled (a) in time. It is also possible for two or more steps to occur simultaneously or to overlap to an extent, except in cases where simultaneity or overlapping would be inconsistent with the internal language of the various steps or with other language in the claims.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of applying alternating electric fields to both a first region of interest within a subject's body and a second region of interest within the subject's body, the method comprising:
(a) applying an alternating voltage between (i) a first electrode assembly positioned at a first location on or in the subject's body and (ii) a plurality of second electrode assemblies, each of which is positioned at a respective second location on or in the subject's body,
wherein the first electrode assembly and a first one of the second electrode assemblies are positioned on opposite sides of the first region of interest,
wherein the first electrode assembly and a second one of the second electrode assemblies are positioned on opposite sides of the second region of interest,
wherein the first one of the second electrode assemblies and the second one of the second electrode assemblies are spaced apart from each other by at least 5 cm, and
wherein the alternating voltage has a frequency between 50 kHz and 1 MHz.

2. The method of claim 1, further comprising:
(b) applying an alternating voltage between a third electrode assembly positioned at a third location on or in the subject's body and a fourth electrode assembly positioned at a fourth location on or in the subject's body,
wherein the third electrode assembly is positioned between the first electrode assembly and the first one of the second electrode assemblies, and
wherein the fourth electrode assembly is positioned between the first electrode assembly and the second one of the second electrode assemblies.

3. The method of claim 2, further comprising repeating step (a) and step (b) in an alternating sequence at least 100 times.

4. The method of claim 1, further comprising:
(b) applying an alternating voltage between a third electrode assembly positioned at a third location on or in the subject's body and a fourth electrode assembly positioned at a fourth location on or in the subject's body,
wherein the third electrode assembly is positioned radially between the first electrode assembly and the first one of the second electrode assemblies, and
wherein the fourth electrode assembly is positioned radially between the first electrode assembly and the second one of the second electrode assemblies.

5. The method of claim 4, further comprising repeating step (a) and step (b) in an alternating sequence at least 100 times.

6. The method of claim 1, further comprising:
(b) applying an alternating voltage between (i) a third electrode assembly positioned at a third location on or in the subject's body and (ii) a plurality of fourth electrode assemblies, each of which is positioned at a respective fourth location on or in the subject's body,
wherein the third electrode assembly is positioned between the first one of the second electrode assemblies and the second one of the second electrode assemblies, and
wherein the first electrode assembly is positioned between a first one of the fourth electrode assemblies and a second one of the fourth electrode assemblies.

7. The method of claim 6, further comprising repeating step (a) and step (b) in an alternating sequence at least 100 times.

8. The method of claim 1, further comprising:
(b) applying an alternating voltage between (i) a third electrode assembly positioned at a third location on or in the subject's body and (ii) a plurality of fourth electrode assemblies, each of which is positioned at a respective fourth location on or in the subject's body,
wherein the third electrode assembly is positioned radially between the first one of the second electrode assemblies and the second one of the second electrode assemblies, and
wherein the first electrode assembly is positioned radially between a first one of the fourth electrode assemblies and a second one of the fourth electrode assemblies.

9. The method of claim 8, further comprising repeating step (a) and step (b) in an alternating sequence at least 100 times.

10. The method of claim 1, wherein the subject's body includes a third region of interest,
wherein the first electrode assembly and a third one of the second electrode assemblies are positioned on opposite sides of the third region of interest, and
wherein all of the second electrode assemblies are spaced apart from each other by at least 5 cm.

11. The method of claim 10, wherein all of the second electrode assemblies are spaced apart from each other by at least 8 cm.

12. The method of claim 1, wherein the first one of the second electrode assemblies and the second one of the second electrode assemblies are spaced far enough apart from each other to create a low-intensity-field zone between the first region of interest and the second region of interest.

13. The method of claim 1, wherein the first one of the second electrode assemblies and the second one of the second electrode assemblies are spaced apart from each other by at least 8 cm.

14. The method of claim 1, further comprising, prior to step (a):
positioning the first electrode assembly at the first location on or in the subject's body; and
positioning each of the plurality of second electrode assemblies at the respective second location on or in the subject's body.

15. The method of claim 1, wherein the alternating voltage has a frequency between 75 kHz and 300 kHz.

16. An apparatus for applying alternating electric fields to both a first region of interest within a subject's body and a second region of interest within the subject's body, the apparatus comprising:
an AC signal generator that operates at a frequency between 50 kHz and 1 MHz, wherein the AC signal generator applies a first output voltage across a first output pin and a second output pin;
a first electrode assembly that is configured to adhere to the subject's body and is connected to the first output pin via a first cable; and
a plurality of second electrode assemblies, each of which is configured to adhere to the subject's body and is connected to the second output pin via a respective one of a plurality of second cables, wherein all of the second electrode assemblies are wired in parallel,
wherein the plurality of second cables are configured so that the plurality of second electrode assemblies can be spaced apart from each other by at least 5 cm.

17. The apparatus of claim 16, wherein the AC signal generator applies a second output voltage across a third output pin and a fourth output pin, and wherein the apparatus further comprises:
a third electrode assembly that is configured to adhere to the subject's body and is connected to the third output pin via a third cable; and
a fourth electrode assembly that is configured to adhere to the subject's body and is connected to the fourth output pin via a fourth cable, and
wherein the AC signal generator is configured to (a) apply the first output voltage across the first output pin and the second output pin, (b) apply the second output voltage across the third output pin and the fourth output pin, and repeat (a) and (b) in an alternating sequence at least 100 times.

18. The apparatus of claim 16, wherein the AC signal generator applies a second output voltage across a third output pin and a fourth output pin, and wherein the apparatus further comprises:
a third electrode assembly that is configured to adhere to the subject's body and is connected to the third output pin via a third cable; and
a plurality of fourth electrode assemblies, each of which is configured to adhere to the subject's body and is connected to the fourth output pin via a respective one of a plurality of fourth cables, wherein all of the fourth electrode assemblies are wired in parallel,
wherein the plurality of fourth cables are configured so that the plurality of fourth electrode assemblies can be spaced apart from each other by at least 5 cm, and
wherein the AC signal generator is configured to (a) apply the first output voltage across the first output pin and the second output pin, (b) apply the second output voltage across the third output pin and the fourth output pin, and repeat (a) and (b) in an alternating sequence at least 100 times.

19. The apparatus of claim 16, wherein the AC signal generator operates at a frequency between 75 kHz and 300 kHz.
